(19) United States
(12) Patent Application Publication
(10) Pub. No.: US 2026/0247740 A1
(43) Pub. Date: Aug. 20, 2026

(54) Solar Panel Recycling Using Deep Eutectic Solvents

(71) Applicant: Battelle Memorial Institute, Columbus, OH (US)

(72) Inventors: Morgan V. Evans, Columbus, OH (US); Karl Rickert, Worthington, OH (US); Erica S. Howard, Upper Arlington, OH (US); Abby Dietrich, Columbus, OH (US); Jacob L. Lilly, Dublin, OH (US); Albert J. Darling, Bexley, OH (US)

(21) Appl. No.: 19/541,588

(22) Filed: Feb. 17, 2026

Related U.S. Application Data
(60) Provisional application No. 63/759,404, filed on Feb. 17, 2025.
(60) Provisional application No. 63/760,178, filed on Feb. 19, 2025.

(51) Int. Cl. H10F 71/00; C01B 33/037; C07D 487/04; C22B 7/00; H10F 19/80; H10F 77/00; H10F 77/12

(52) U.S. Cl. CPC H10F 71/121; C01B 33/037; C07D 487/04; C22B 7/006; H10F 19/804; H10F 77/126; H10F 77/937

(57) ABSTRACT

The present disclosure is directed at solar panel recycling utilizing deep eutectic solvents. More specifically, solar panels selected from various stages of a solar panel recycling pathway may now be exposed to deep eutectic solvents to selectively recover one or more metal elements employed in solar panel construction.

### STATEMENT REGARDING FEDERALLY SPONSORED RESEARCH OR DEVELOPMENT

This invention was made with government support under Contract Number 49100424C0006, awarded by the National Science Foundation (NSF). The government has certain rights in the invention.

### FIELD

The present disclosure is directed at solar panel recycling utilizing deep eutectic solvents. More specifically, solar panels selected from various stages of a solar panel recycling pathway may now be exposed to deep eutectic solvents to selectively recover one or more metal elements employed in solar panel construction.

### BACKGROUND

It has been reported that waste from end-of-life solar panels will present opportunities to recover valuable materials through recycling. According to the International Renewable Energy Agency, in the report entitled End-Of-Life Management/Solar Photovoltaic Panels (June 2016), solar photovoltaic (PV) deployment has grown significantly since the early 2000s, and at the end of 2016, cumulative global PV waste streams were expected to reach 43,500 to 250,000 metric tons. It was suggested that PV recycling would be essential in the world's transition to a sustainable, economically viable and increasingly renewable-based energy future.

### SUMMARY

A method for recovering one or more metals from a mixture of metals from a recycled solar panel comprising supplying a solar panel containing a plurality of metals and milling said solar panel to a particle size diameter in the range of 1.0 μm to 10 mm and length up to 10.0 mm; placing the solar panel particles into a deep eutectic solvent (DES) and selectively dissolving one or more of the plurality of metals; and recovering one or more of the dissolved metals. The one or more metals may include silver (Ag), aluminum (Al), copper (Cu), nickel (Ni), thallium (Tl), titanium (Ti) and tin (Sn). Optionally, one may first remove certain portions of the recycled solar panels before treatment with the deep eutectic solvent, such as removal of glass, the metallic solar panel frame (e.g. aluminum), selected electronic components and/or the solar panel junction box.

### BRIEF DESCRIPTION OF THE FIGURES

FIG. 1 shows the percentage of material dissolved in the DES of choline chloride and ethylene glycol (ChCl:EG).

FIG. 2 shows the percentage of material dissolved in the DES of choline chloride and oxalic acid with 2.0 ml of 30% H₂O₂ in 10 ml of the DES.

### DETAILED DESCRIPTION OF THE PREFERRED EMBODIMENTS

Solar panels suitable for recycling herein typically include silicon solar cells, a metal frame (typically aluminum), glass sheet, polymeric material (adhesive/backsheet), 12V wire, bus wire and a junction box. The silicon solar cells are employed to convert sunlight into electricity relying upon the photovoltaic effect. Reference to a recycled solar panel includes solar panels that have been employed for electricity generation and/or solar panels that may be identified as unsuitable for distribution or installation. Preferably, the crystalline silicon (c-Si) solar panels utilized herein for recycling undergo separation of one or more of the metallic (e.g., aluminum) frame, selected electronic components and/or junction box, followed by mechanical crushing, shredding and/or milling. For the ensuing recycling herein using deep eutectic solvents, it is contemplated that the milled solar panels are preferably reduced to a particle size having a diameter in the range of 1.0 μm to 10000.0 μm (10.0 mm) and up to 10.0 mm in length, including all values and increments therein. Accordingly, the diameter may range from 1.0 μm to 9.0 mm, or 1.0 μm to 8.0 mm, or 1.0 μm to 7.0 mm, or 1.0 μm to 6.0 mm, or 1.0 μm to 5.0 mm, or 1.0 μm to 4.0 mm, or 1.0 μm to 3.0 mm, or 1.0 μm to 2.0 mm, or 1.0 μm to 1.0 mm. In addition, the length may range from 1.0 mm to 10.0 mm, or 1.0 mm to 9.0 mm, or 1.0 mm to 8.0 mm, or 1.0 mm to 7.0 mm, or 1.0 mm to 6.0 mm or 1.0 mm to 5.0 mm, or 1.0 mm to 4.0 mm, or 1.0 mm to 3.0 mm, or 1.0 mm to 2.0 mm.

Optionally, one may first elect to remove silicon (Si) from the milled solar panels. Preferably, the silicon may be removed by use of the Czochralski crystal pulling method. Accordingly, it is contemplated that the milled solar panel material may be placed into a crucible and melted by resistance or radio-frequency heaters. After such solar panel feed material is melted, a seed crystal of silicon may be introduced into the free melt surface and a melt meniscus is formed. Then, upon remelting of the dipped seed crystal, the seed is withdrawn (pulled) from the melt (typically with rotation) and the melt crystallizes at the seed interface by forming a new silicon crystal portion. Accordingly, silicon may be preferably removed from the molten solar panel material. It is contemplated that 70.0% by weight (wt.) or more of the silicon may be removed from the molten solar panel material by this procedure. Accordingly, it is contemplated that 70% (wt.) to 100% (wt.) of the silicon may be removed from the molten solar panel material, including all values and increments therein. For example, 70.0% (wt.) or more, 75.0% (wt.) or more, 80.0% (wt.) or more, 85.0% (wt.) or more, 90.0% (wt.) or more or 95.0% (wt.) or more of the silicon may be removed.

The milled solar panel material (optionally with the silicon removed according to the Czochralski crystal pulling procedure noted above) may then be subject to selective removal of the residual metals therein. It is contemplated that the residual metals in the milled solar panel material may therefore include the following seven (7) metal elements: silver (Ag), aluminum (Al), copper (Cu), nickel (Ni), thallium (Tl), titanium (Ti) and tin (Sn). Such mixture of residual metals are then contemplated to be treated with a deep eutectic solvent (DES) system. Optionally, the DES system may include hydrogen peroxide, namely up to 2.0 ml of 9-10 molar (M) hydrogen peroxide in 10 ml of the DES.

Reference to a deep eutectic solvent system is reference to a mixture of two or more components that form a liquid with a lower melting point than the individual components. Generally, they can therefore include solutions of Lewis or Bronsted acids and bases which form the deep eutectic mixture. It is contemplated that treatment of the seven (7) element mixture of Ag, Al, Cu, Ni, Tl, Ti and Sn, sourced from the solar panels, with a deep eutectic solvent system, can provide the ability to selectivity dissolve and remove one or more of such elements from the mixture, at a purity of 70.0% or more.

Reference to such metallic purity corresponds to the mass of pure substance to the total mass of the sample and calculating the percentage. In the broader context of the present disclosure, it is contemplated that the one or more of the residual metals that are now removed from the milled solar panels can be recovered in the purity range of 70.0% to 100%, including all values and increments therein. Accordingly, the particular level of purity of the one or more residual metallic elements removed from the mixture of Ag, Al, Cu, Ni, Tl, Ti and Sn may be 70.0% or more, 75.0%, or more 80.0% or more, 85.0% or more, 90.0% or more and 95.0% or more.

In addition, when treating the milled solar panels containing a mixture of Ag, Al, Cu, Ni, Tl, Ti and Sn, to selectively dissolve one or more of such elements, it should be noted that temperature control may be utilized to selectively dissolve one of such elements and not the others. Once dissolved the metals can be recovered at the purity levels noted above by promoting precipitation, which can be preferably achieved with acids, bases, or complexing agents (e.g. metal chelators). Alternatively, electrochemical methods may be used to selectively deposit specific dissolved metals, which can take place in the initial deep eutectic solvent used to dissolve the metals, or where the dissolved metals may be placed in other solvents, such as a different eutectic solvent, an aqueous electrolyte, or an ionic solvent (a salt in a liquid state at ambient conditions).

There are several preferred deep eutectic solvent systems that are contemplated herein for use for the above-described solar panel recycling. They are as follows:
(1) Choline chloride [(CH₃)₃NCH₂CH₂OH]⁺Cl⁻/oxalic acid [HO—C(═O)—C(═O)—OH]. The choline chloride/oxalic acid deep eutectic solvent is targeted for the selective removal of Cu, Ag and Al from the mixture of Ag, Al, Cu, Ni, Tl, Ti and Sn. Preferably, the choline chloride/oxalic acid deep eutectic combination is present at a 1:1 mol/mol ratio, optionally in the presence of a selected amount of hydrogen peroxide (H₂O₂) to improve the dissolution of the Cu, Ag and Al. Other oxidants contemplated for use include copper (II) chloride or iron (III) chloride hexahydrate.
(2) Calcium chloride hexahydrate [CaCl₂*6H₂O]/ethylene glycol [HOCH₂CH₂OH]. The calcium chloride hexahydrate/ethylene glycol deep eutectic solvent is targeted for the selective removal of Cu and Ni from the mixture of Ag, Al, Cu, Ni, Tl, Ti and Sn. Preferably, the calcium chloride hexahydrate and ethylene glycol deep eutectic combination is present at a 1:1 mol/mol ratio.
(3) Choline chloride [(CH₃)₃NCH₂CH₂OH]⁺Cl⁻/ethylene glycol [HOCH₂CH₂OH]. The choline chloride/ethylene glycol deep eutectic solvent is targeted for the selective removal of Cu, Ni and Sn from the mixture of Ag, Al, Cu, Ni, Tl, Ti and Sn. Preferably, the choline chloride and ethylene glycol combination is present at a 1:2 mol/mol ratio.
(4) Betaine [(CH₃)₃N⁺CH₂CO₂⁻]/malic acid [HO₂CCH(OH)CH₂CO₂H]/ethylene glycol [HOCH₂CH₂OH]. The betaine/malic acid/ethylene glycol deep eutectic solvent is targeted for the selective removal of Cu from the mixture of Ag, Al, Cu, Ni, Tl, Ti and Sn. Preferably, the betaine/malic acid/ethylene glycol deep eutectic combination is present at a 1:1:2 mol/mol/mol ratio.
(5) Choline chloride [(CH₃)₃NCH₂CH₂OH]⁺Cl⁻/malonic acid [CH₂(COOH)₂]. The choline chloride/malonic acid deep eutectic solvent is targeted for the selective removal of Cu, Ni and Ti from the mixture of Ag, Al, Cu, Ni, Tl, Ti and Sn. Preferably, the choline chloride/malonic acid deep eutectic combination is present at a 1:1 mol/mol ratio.

(6) Choline chloride [(CH₃)₃NCH₂CH₂OH]⁺Cl⁻/urea [H₂N—CO—NH₂]. The choline chloride/urea deep eutectic solvent is targeted for the selective removal of Cu, Ni and Ti from the mixture of Ag, Al, Cu, Ni, Tl, Ti and Sn. Preferably, the choline chloride/urea deep eutectic combination is present at a 1:2 mol/mol ratio.

In addition, it is also contemplated herein that the deep eutectic solvents selectively containing one or more dissolved metals from the mixture of Ag, Al, Cu, Ni, Tl, Ti and Sn may be utilized in an electrodeposition (ED) process. Namely electrodeposition of one or more of the dissolved metals in the deep eutectic solvent is treated with an electric current in which case they then would form a metal or metal oxide or ceramic coating on an electrode. More specifically, the one or more dissolved metals in the deep eutectic solvent serves as the solution between a cathode and anode, an electric current is applied and oxidation and reduction reactions can occur and a solid metal or metal oxide film may then be deposited on one of the electrodes. Accordingly, the electrodeposition process is configured to selectively deposit one or more of the dissolved metals in the form of a metal or metal oxide from the mixture of Ag, Al, Cu, Ni, Tl, Ti and Sn recovered from solar panels.

### WORKING EXAMPLES

Example 1: Recycled solar panel particulate was produced at the following particle size range: 1 μm to 100 μm; 2. 1 μm to 500 μm; 3. 10 μm to 5 mm; and 4. 5 μm to 10 mm. A deep eutectic solvent was prepared from the combination of choline chloride/ethylene glycol. The percentage of metal (Cu) removed and dissolved in this deep eutectic solvent after treatment of the indicated recycled solar panel particulate particle size distributions was as follows: Cu: 20% (1); Cu: 70% (2); Cu 20% (3).

Example 2: Recycled solar panel particulate was again produced at the following particle size range: 1 μm to 100 μm; 2. 1 μm to 500 μm; 3. 10 μm to 5 mm; and 4. 5 μm to 10 mm. A deep eutectic solvent was prepared from the combination of choline chloride/oxalic acid in the presence of 9-10 M hydrogen peroxide (1-2 ml/10 ml of the deep eutectic solvent). The percentage of metal removed and dissolved in this deep eutectic solvent after treatment of the indicated recycled solar panel particulate particle size distributions was as follows: Cu: 22% (1); Cu: 65% (2); Cu 40% (3).

Attention is next directed to Table 1 below, which shows the use of deep eutectic solvent #1 of choline chlorine and oxalic acid with 2.0 ml of 30% hydrogen peroxide in 10 ml of the DES and deep eutectic solvent #2 of choline chloride and ethylene glycol for recovery of the identified metals. The recovery range for nitric acid may be viewed as a control example where nitric acid was used to dissolve the solar panel particles.

TABLE 1
Deep Eutectic Solvent Application To Solar Panels

| | | | | | Recovery |
|---|---|---|---|---|---|
| | | Concentration | Recovery | Recovery | Range for |
| | Contaminant/ | Range in | Range for | Range for | Nitric Acid |
| Metal | Desirable | Samples (ppm) | DES #1 | DES #2 (%) | (%) |
| Sodium | Contaminant | 50,000-100,000 | 5% ± 4% | 1% ± 1% | 0% ± 0% |
| Aluminum | Desirable | 2,000-13,000 | 122% ± 53% | 0% ± 0% | 5% ± 2% |
| Silicon | Desirable | 200,000-500,000 | 0% ± 0% | 0% ± 0% | 0% ± 0% |
| Chromium | Desirable | 80-500 | 31% ± 44% | 0% ± 0% | 0% ± 0% |
| Manganese | Desirable | 20-100 | 61% ± 43% | 0% ± 0% | 0% ± 0% |
| Iron | Contaminant | 1,000-8,000 | 46% ± 34% | 0% ± 0% | 0% ± 0% |
| Copper | Desirable | 1,500-4,500 | 36% ± 18% | 27% ± 19% | 0% ± 0% |
| Lead | Contaminant | 1,000-2,500 | 84% ± 9% | 9% ± 2% | 3% ± 1% |
| Calcium | Contaminant | 15,000-34,000 | 0% ± 0% | 0% ± 0% | 0% ± 0% |
| Magnesium | Contaminant | 3,000-6,000 | 0% ± 0% | 0% ± 0% | 0% ± 0% |

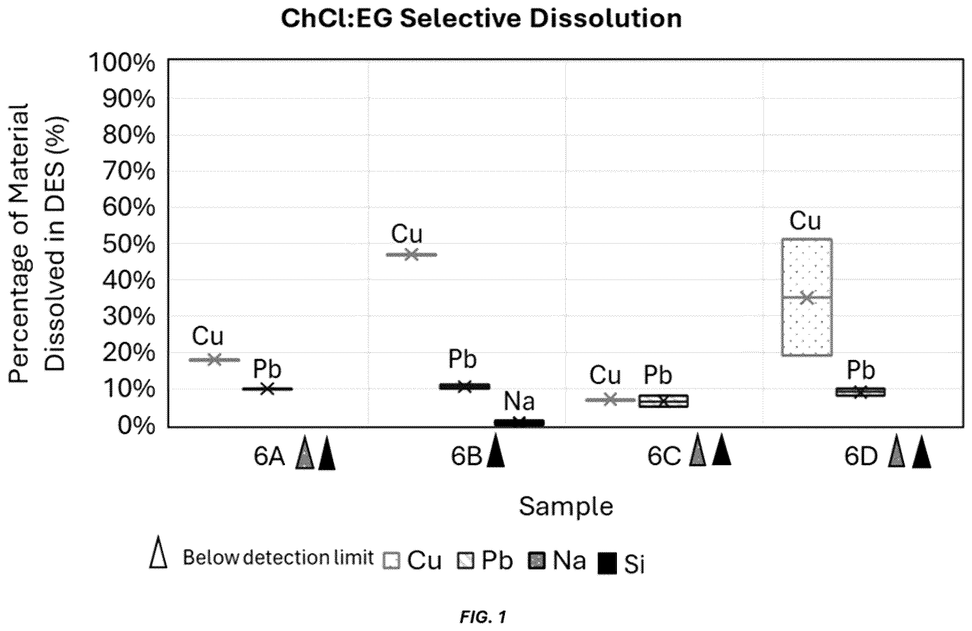

Attention is next directed to FIG. 1 which shows the percentage of material dissolved in the DES of choline chloride and ethylene glycol (ChCl:EG). Samples 6A-6D are different particle sizes of recycled panel samples after removal of glass. Sample 6A had a particle size of 10 μm to 500 μm, sample 6B had a particle size of 25 μm to 250 μm, sample 6C had a particle size of 300 μm to 5 mm and sample 6D had a particle size of 5 μm to 40 μm. The mean is indicated at the “X” on the graph.

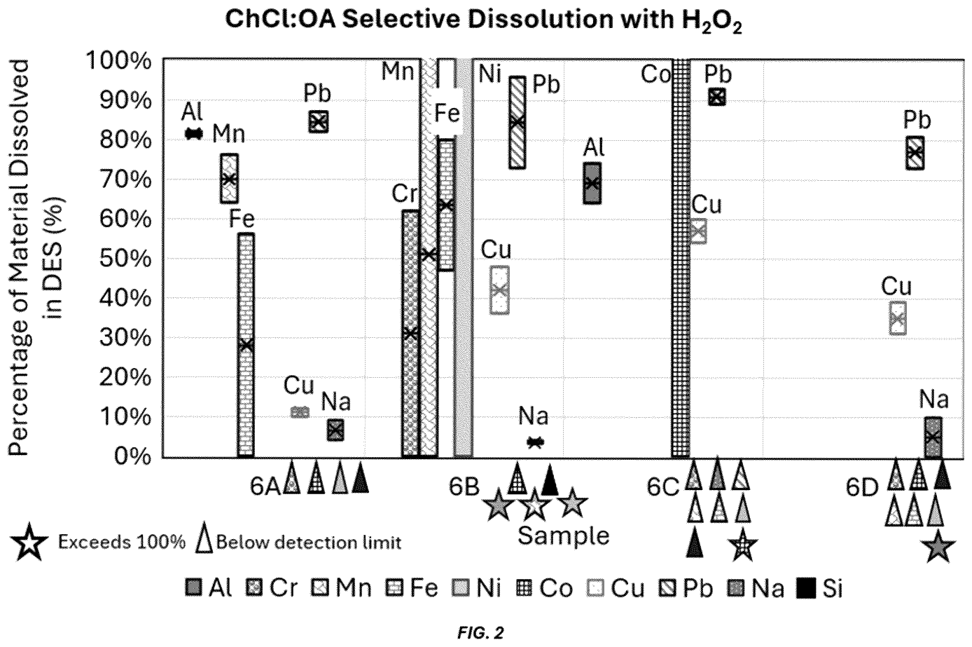

FIG. 2 shows the percentage of material dissolved in the DES (%) of choline chloride and oxalic acid (ChCl:OA) with 2.0 ml of 30% H₂O₂ in 10 ml of the DES. Sample 6A had a particle size of 10 μm to 500 μm, sample 6B had a particle size of 25 μm to 250 μm, sample 6C had a particle size of 300 μm to 5 mm and sample 6D had a particle size of 5 μm to 40 μm. The mean is indicated at the “X” on the graph.

1. A method for recovering one or more metals from a mixture of metals from a recycled solar panel comprising:
a. supplying a solar panel containing a plurality of metals and milling said solar panel to a particle size diameter in the range of 1.0 μm to 10.0 mm and length up to 10.0 mm;
b. placing said solar panel particles into a deep eutectic solvent and selectively dissolving one or more of said plurality of metals; and
c. recovering said one or more dissolved metals.

2. The method of claim 1 wherein said solar panel includes a metallic frame, electronic components and a junction box, and prior to milling of said solar panels, one or more of said metallic frame, electronic components and/or junction box are removed from said solar panel.

3. The method of claim 1 wherein said solar panel contains silicon, and prior to placing said solar panel particles into said deep eutectic solvent all or a portion of said silicon is removed.

4. The method of claim 3 wherein 70.0% (wt.) or more of silicon is removed from said solar panel particles.

5. The method of claim 1 wherein said one or more metals is recovered at a purity of 70% or more.

6. The method of claim 1 wherein said solar panel containing a plurality of metals contains a mixture of Ag, Al, Cu, Ni, Tl, Ti and Sn.

7. The method of claim 6 wherein recovering one or more dissolved metals comprises recovery of Ag.

8. The method of claim 6 wherein recovering one or more dissolved metals comprises recovery of Al.

9. The method of claim 6 wherein recovering one or more dissolved metals comprises recovery of Cu.

10. The method of claim 6 wherein recovering one or more dissolved metals comprises recovery of Ni.

11. The method of claim 6 wherein recovering one or more dissolved metals comprises recovery of Tl.

12. The method of claim 6 wherein recovering one or more dissolved metals comprises recovery of Ti.

13. The method of claim 6 wherein recovering one or more dissolved metals comprises recovery of Sn.

14. The method of claim 1 wherein recovering one or more dissolved metals comprises treating said deep eutectic solvent containing one or more of said dissolved plurality of metals with an electric current and forming a metal or metal oxide coating.

15. The method of claim 1 wherein said deep eutectic solvent comprises choline chloride and oxalic acid.

16. The method of claim 1 wherein said deep eutectic solvent comprises calcium chloride hexahydrate and ethylene glycol.

17. The method of claim 1 wherein said deep eutectic solvent comprises choline chloride and ethylene glycol.

18. The method of claim 1 wherein said deep eutectic solvent comprises betaine, malic acid and ethylene glycol.

19. The method of claim 1 wherein said deep eutectic solvent comprises choline chloride and malonic acid.

20. The method of claim 1 wherein said deep eutectic solvent comprises choline chloride and urea.

21. The method of claim 1 where said deep eutectic solvent includes one of hydrogen peroxide, copper (II) chloride or iron (III) chloride hexahydrate.
